(19) United States
(12) Patent Application Publication
(10) Pub. No.: US 2018/0366211 A1
(43) Pub. Date: Dec. 20, 2018

(54) BEHAVIOR TRAINED CLINICAL SUPPORT

(71) Applicant: KONINKLIJKE PHILIPS N.V., EINDHOVEN (NL)

(72) Inventors: Lin Yang, Chandler, AZ (US); Minnan Xu, Cambridge, MA (US); Stijn De Waele, Millwood, NY (US); Rony Calo, Amsterdam (NL)

(21) Appl. No.: 16/061,372

(22) PCT Filed: Dec. 20, 2016
(86) PCT No.: PCT/IB2016/057802

§ 371 Date: Jun. 12, 2018

Related U.S. Application Data
(60) Provisional application No. 62/270,152, filed on Dec. 21, 2015.

(51) Int. Cl. G16H 10/40; G16H 50/20; G16H 50/70; G06N 7/00; G06K 9/62; G06F 17/18

(52) U.S. Cl. CPC G16H 10/40; G16H 50/20; G06F 17/18; G06N 7/005; G06K 9/6269; G16H 50/70

(57) ABSTRACT

Systems for user behavior-trained clinical support. Features of the present invention monitor users to track their behavior while using medical devices/software. This information is combined with physiological parameters of patients to train new clinical decision support (CDS) algorithms to provide improvement in deterioration detection, alarm management, and decision and navigation support.

### TECHNICAL FIELD

This invention generally relates to training support systems and, more particularly, to training support systems based on user behavior.

### BACKGROUND

Medical personnel or the like frequently interact with software and various medical devices. Learning how these medical personnel interact with these software platforms and devices, as well as their expectations of new technologies, may be helpful in creating more effective software platforms and medical devices.

It is often difficult, however, to gather helpful information regarding these medical personnel interactions. Current techniques to gather this type of information may involve interviews with medical personnel. However, conducting these interviews may be time consuming, labor intensive, and takes medical personnel away from their medical duties.

There are also typically variations in usage among personnel employed by different healthcare institutions and treating different kinds of patients. Collaborating with or interviewing a subgroup of professionals can yield biased results that are not representative of all healthcare institutions and medical personnel. On the other hand, interviewing larger groups of medical personnel and tracking their daily workflows may be difficult if not impossible.

Even with close collaboration, efficient communication with medical personnel and healthcare institutions still cannot be guaranteed. For example, existing clinical decision support (CDS) tools inevitably generate noise or redundant information that can be distracting rather than helpful. Also, there may be areas not recognized (i.e., not currently monitored) where CDS tools could intervene to support patient care.

A need exists, therefore, for improved clinical decision support systems.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

In one aspect, embodiments of the invention relate to a non-transitory computer-readable medium having instructions stored thereon for performing a method of behavior-trained deterioration detection, the instructions operative on one or more processors to perform operations comprising: for at least one patient experiencing an episode, retrieving records of medical personnel actions preceding and following the episode for the at least one patient from at least one records database, using at least some of the retrieved action records to determine whether the condition of the at least one patient is at risk of deterioration; for at least one other patient experiencing a similar episode, retrieving records of physiological parameters preceding and following the similar episode for at least one other patient from at least one parameter database, categorizing the at least one other patient into a control group or a positive group using at least some of the retrieved action records; and training at least one algorithm for deterioration detection utilizing the retrieved physiological parameters for at least one patient in the positive group.

In some embodiments of the computer readable medium for deterioration detection, the at least one records database is at least one of an electronic medical record (EMR) system and a clinical decision support (CDS) system.

In some embodiments of the computer readable medium for deterioration detection, the at least one parameter database is at least one of an electronic medical record (EMR) system and a clinical decision support (CDS) system.

In some embodiments of the computer readable medium for deterioration detection, the duration of the period preceding and following the episode for the retrieval of action records depends on the episode.

In some embodiments of the computer readable medium for deterioration detection, the duration of the period preceding and following the similar episode for the retrieval of parameter records depends on the episode.

In some embodiments of the computer readable medium for deterioration detection, the training is selected from the group consisting of regression, decision trees, random forest, vector support machine, and Bayesian methods.

In another aspect, embodiments of the invention relate to a non-transitory computer-readable medium having instructions stored thereon for performing a method of behavior-trained alarm management, the instructions operative on one or more processors to perform operations comprising: for at least one indicator, retrieving records of medical personnel actions preceding and following the indicator from at least one records database; using at least some of the retrieved action records to determine whether the condition of at least one patient is at risk of deterioration; for at least one other patient experiencing a similar indicator, retrieving records of physiological parameters preceding and following the similar indicator for at least one other patient from at least one parameter database; categorizing the similar indicator as a true positive, false positive, true negative, or false negative using at least some of the retrieved action records; and training at least one algorithm utilizing the categorized indicator.

In some embodiments of the computer readable medium for alarm management, the indicator is an alarm.

In some embodiments of the computer readable medium for alarm management, the at least one records database is at least one of an electronic medical record (EMR) system and a clinical decision support (CDS) system.

In some embodiments of the computer readable medium for alarm management, the at least one parameter database is at least one of an electronic medical record (EMR) system and a clinical decision support (CDS) system.

In some embodiments of the computer readable medium for alarm management, the duration of the period preceding and following the indicator for the retrieval of action records depends on the episode.

In some embodiments of the computer readable medium for alarm management, the duration of the period preceding and following the similar indicator for the retrieval of parameter records depends on the episode.

In some embodiments of the computer readable medium for alarm management, the training is selected from the group consisting of regression, decision trees, random forest, vector support machine, and Bayesian methods.

In yet another aspect, embodiments of the present invention relate to a non-transitory computer-readable medium having instructions stored thereon for performing a method of behavior-trained decision support, the instructions operative on one or more processors to perform operations comprising: for at least one event, retrieving records of medical personnel actions preceding and following the event from at least one records database; and providing guidance to medical personnel using at least some of the retrieved action records.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

The invention and embodiments thereof will be better understood when the following detailed description is read in conjunction with the accompanying drawing figures:

FIG. 1 schematically illustrates a system for behavior-trained clinical support in accordance with one embodiment of the invention;

FIG. 2 illustrates several types of the input/output (I/O) devices 102 of FIG. 1 in accordance with one embodiment of the invention;

FIG. 3 illustrates a series of steps performed by the deterioration detection module 114 of FIG. 1 in accordance with one embodiment of the invention;

FIG. 4 depicts a flowchart of a method of behavior-trained deterioration detection in accordance with one embodiment of the invention;

FIG. 5 illustrates a series of steps performed by the alarm management module of FIG. 1 in accordance with one embodiment of the invention;

FIG. 6 presents a flowchart of a method of behavior-trained alarm management in accordance with one embodiment of the invention;

FIG. 7 illustrates a series of steps performed by the alarm management module 116 of FIG. 1 in accordance with another embodiment of the invention;

FIG. 8 illustrates a series of steps performed by the decision and navigation support module 118 of FIG. 1 in accordance with one embodiment of the invention;

FIG. 9 depicts a flowchart of a method of behavior-trained decision support in accordance with one embodiment of the invention; and

FIG. 10 illustrates an exemplary application of the decision and navigation support module of FIG. 1.

In the drawings, like reference characters generally refer to corresponding parts throughout the different views. Elements are not necessarily drawn to scale, emphasis instead being placed on the principles and concepts of operation.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein;

rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

Having knowledge of how medical personnel interact with medical devices and software may help design more effective and user-friendly medical devices and software. This may in turn lead to higher quality patient care. A variety of patient care-related applications may benefit from the features of the invention.

For example, deterioration detection tools typically monitor patients' vital signs and other physiological parameters, calculate a score using the monitored parameters, and trigger an alarm if the score exceeds a certain threshold. Having an in-depth understanding of the medical problem and the healthcare institution workflow (e.g., how medical personnel react to certain patient symptoms and patient physiology) may also be used to improve deterioration detection.

Alarm systems are another important component of deterioration detection. These systems may generate a score based on physiological parameters, and an indicator (e.g., an alarm) may be issued if the score is above (or below) a predetermined threshold. For system designers, it may be important to know how to balance the sensitivity and specificity of the alarm system. Having an in-depth understanding of the users' experiences, their tolerance to false alarms, and their preferences as to how the system should be integrated into their workflow may therefore improve alarm management.

Another application may involve the integration of multiple deterioration detection methods to provide decision and navigation support (e.g., steps to be taken by medical personnel). For system designers, it may be important to know how medical personnel interact with each other, the particular tests they order for particular medical conditions, the medications they typically prescribe for certain conditions, and the like.

Features of the present invention therefore analyze individual users' behavior as it relates to multiple medical devices and/or software platforms. The ways users respond to alarms, how they treat certain patient conditions, and their sequential movement across multiple platforms all reflect their medical expertise. Mining this behavior data, along with patients' physiological data, may lead to a better understanding of users' medical knowledge which can be leveraged to train new CDS methods.

It is also contemplated that embodiments of the present invention may be used in other types of applications, such as those in the military, retail, e-commerce, finance, transportation, or any type of data-driven application that may benefit from the features of the present invention. For the sake of simplicity, however, the features of the present invention will be described as being implemented in healthcare institutions. The term “user” may refer to any type of medical personnel such as doctors, physicians, nurses, physician assistants, caregivers, receptionists, or any other interested party providing healthcare.

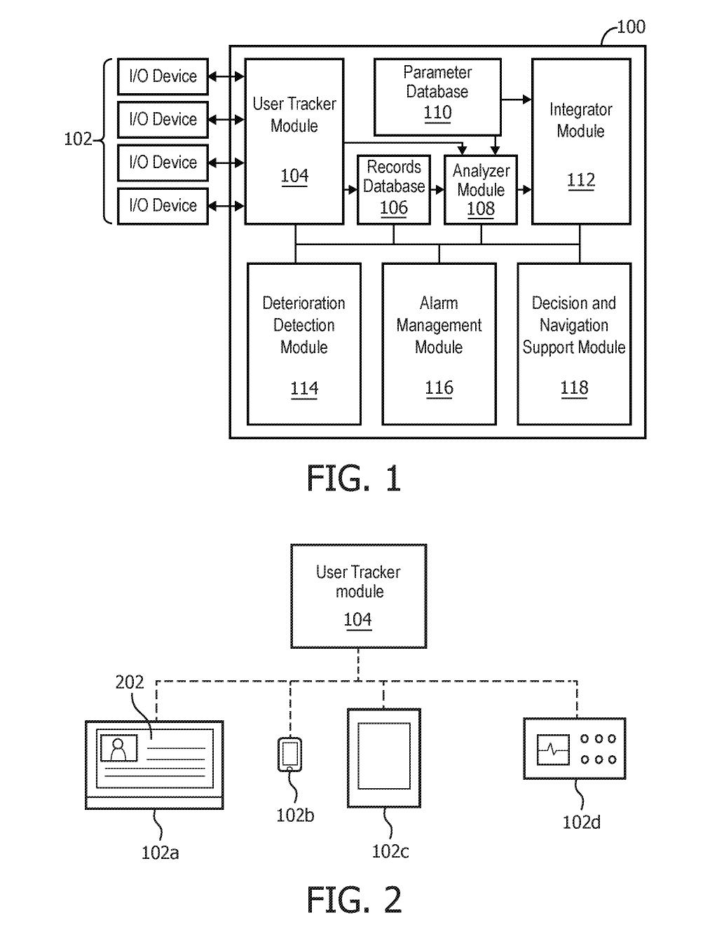

FIG. 1 schematically illustrates various components of the behavior-trained clinical support system 100 in accordance with one embodiment of the invention. Medical personnel or other types of users may interact with a plurality of input/output (I/O) devices 102. The information entered into the I/O devices 102 (along with the user's behavior while interacting with the I/O devices 102) may be monitored by the user tracker module 104.

Information regarding the user's behavior and actions while interacting with the I/O devices 102 may be stored in at least one records database 106 and analyzed by an analyzer module 108 when appropriate. Information regarding the user's behavior may also be communicated directly to the analyzer module 108.

Information regarding patient physiological parameters may be stored in at least one parameter database 110. The integrator module 112 may integrate information regarding thousands of users' behavior with patients' physiological parameters from the parameter database 110.

Outputs of the integrator module 112 may then be used train various CDS platforms, such as, e.g., a deterioration detection module 114, an alarm management module 116, a decision and navigation support module 118, etc.

The I/O devices 102 illustrated in FIG. 1 may be any type of device that can receive or otherwise monitor information regarding a user's behavior (e.g., their actions). FIG. 2 illustrates different types of I/O devices 102 that users often interact with. These may include laptops 102a (as well as desktop computers), mobile devices 102b, tablets 102c, or the like, as well as medical devices 102d. These devices 102 may include an interface to enable interaction between users and software platforms.

With continued reference to FIG. 2, medical personnel may interact with these I/O devices 102 to add information to an electronic medical record (EMR) 202 of a patient, enter test results of a patient, order medications, conduct research, etc. This information may be communicated from the I/O devices 102 to the user tracker module 104 via any type of hardwired or wireless connection.

The user tracker module 104 may be any configured processor or the like that can monitor, record, and communicate information regarding the user's behavior. The user tracker module 104 may, for example, monitor medical interventions taken by the user(s). If the user is using a software-related platform, for example, the user tracker module 104 may monitor the buttons or tabs clicked by the user, the pages viewed, the amount of time the user remains on a particular page, messages sent and received regarding a patient, etc.

Information regarding the user's behavior may be stored in at least one records database 106 until ready for analysis by the analyzer module 108. Or, information regarding the user's behavior may be communicated directly to the analyzer module 108 by the user tracker module 104.

The analyzer module 108 may be configured to perform calculations or other processes on the data regarding the user's behavior. In some embodiments, the analyzer module 108 may be configured to calculate various derivatives of the measured behavioral data, including but not limited to simple averages (i.e., a mean(s)), weighted averages, standard deviations, etc. In some embodiments, the analyzer module 108 may be implemented as a single pole filter, a multipole filter, a Kalman filter, etc.

The system 100 may also include at least one parameter database 110. The parameter database 110 may store information relating to physiological parameters of thousands of patients. This information may relate to patient medical history, patient test results, vital sign measurements, etc.

The parameter database 110 may also be in communication with the analyzer module 108. The analyzer module 108 may therefore process or otherwise analyze parameter-related information prior to integration by the integrator module 112. For example, in some embodiments, the analyzer module 108 may be configured to calculate various derivatives of the parameter-related information, including but not limited to simple averages (i.e., a mean(s)), weighted averages, standard deviations, etc.

The output(s) of the analyzer module 108 (or databases) may be communicated to the integrator module 112. The integrator module 112 may integrate the behavior-related information with parameter-related information to evaluate or otherwise train CDS tools. That is, the modules and databases 104-12 can be implemented in or otherwise used to train CDS tools such as the deterioration detection module 114, the alarm management module 116, and/or the decision and navigation support module 118.

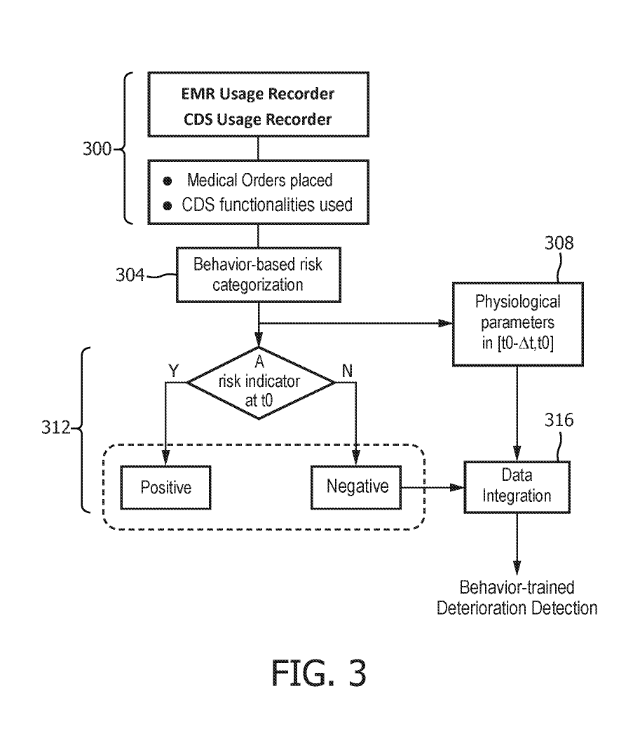

FIG. 3 generally illustrates a series of steps performed by the behavior-trained deterioration detection module 114. Deterioration detection typically utilizes the tools and analytics to determine whether patients have a health-related condition, are at risk of acquiring a health-related condition, or are at risk of spreading a health-related condition.

The user tracker module 104 may monitor or otherwise keep track of the user's behavior when they are treating a patient. As mentioned previously, this may include medical orders placed by the user and/or other user actions interacting with various platforms such as EMR and various CDS tools.

For a particular patient, at any given episode t₀ the analyzer module 108 analyzes the actions taken by the user(s) (e.g., physicians, nurses, or other medical personnel) in a small time window [t_{0-δ}, t_{0+δ}] (Step 300). In this particular embodiment, δ represents the time window preceding/following t₀ in which we assume a user takes actions to treat the patient's condition. These actions could be reviewing certain information, prescribing medications, conducting certain procedures or interventions, or placing any comprehensive medical orders. The variable δ may of course vary based on different applications (and/or may of course vary depending on the particular episode, patient, or condition).

Based on a set of (e.g., predetermined) criteria, the analyzer module 108 may determine if the collection of actions taken in the window [t_{0-δ}, t_{0+δ}] indicate whether a particular patient is at risk of deterioration (or some other health-related condition, depending on the application) at t₀ (Step 304). This determination may result in a “yes” or “no” determination, and may be referred to as behavior-based risk categorization. That is, the categorization is based on the user's behavior.

The integrator module 112 may then obtain physiological parameters in a time window before t₀ [t_{0-Δt}, t₀] from the parameter database 110 (Step 308). Δt represents the time window preceding t₀ in which vital signs and other parameters are measured that may be predictive of the patient's condition at t₀. These physiological parameters may be gathered by a plurality of vital sign sensors (not shown) and, collectively, may relate to physiological parameters of thousands of patients over thousands of episodes. The variable Δt may of course vary depending on the application (and/or depending on the particular episode). The integrator module 112 could be programmed to utilize various types of data mining techniques, such as regression, decision trees, random forest, vector support machine, Bayesian methods, or the like.

The risk categorization made by the analyzer module 108 is used as ground truth to separate patients into a positive group (patients with a particular condition) and a control group (patients without the particular condition) (Step 312). The integrator module 112 integrates the risk categorization and the information regarding the physiological parameters for future iterations of the deterioration detection module 114 (Step 316). The integrator module 112 could be programmed to utilize various types of data mining techniques, such as regression, decision trees, random forest, vector support machine, and Bayesian methods.

For example, the deterioration detection module 114 can be configured to target specific user actions so that the outcome of the system could be an algorithm trained to use a combination of physiological parameters to detect a certain condition (e.g., respiratory illness). Or, the deterioration detection module 114 can be configured to target more actions, so the outcome of the system can be an algorithm used to detect general deteriorations.

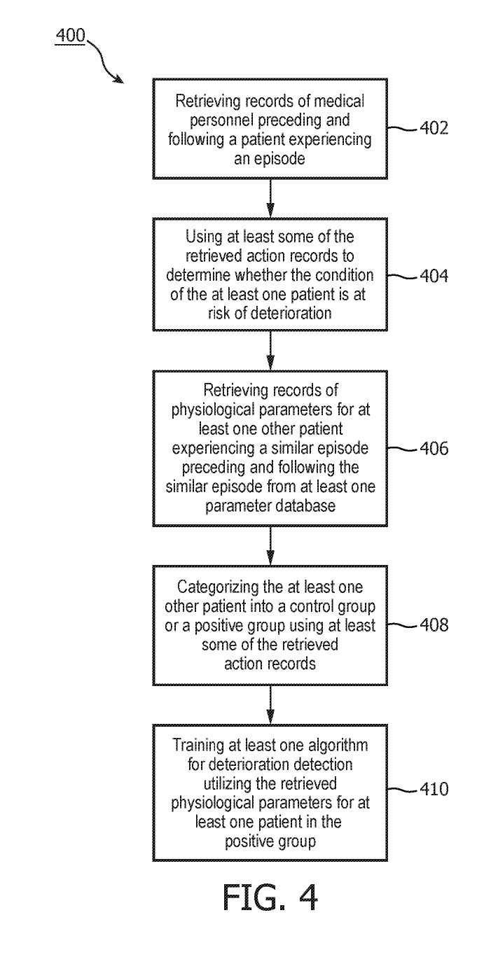

FIG. 4 generally illustrates a method 400 of behavior-trained deterioration detection in accordance with one embodiment of the invention. This method may be performed by any non-transitory computer-readable medium having instructions operative on one or more processors.

For this method 400, assume at least a one patient is experiencing a health condition-related episode. Step 402 involves retrieving records of medical personnel actions preceding and following the episode from at least one records database. As stated previously, these actions may include tests performed by the medical personnel, notes taken by the medical personnel, procedures or interventions conducted by the medical personnel, etc.

Step 404 involves using at least some of the retrieved action records to determine whether the condition of the at least one patient is at risk of deterioration. For example, if these records show that medical personnel ordered a series of medications and/or performed certain procedures, it may be determined that the condition of the at least one patient is at risk of deterioration. This step may result in a “yes” or “no” determination.

Step 406 relates to at least one other patient experiencing a similar episode. Step 406 involves retrieving records of physiological parameters preceding and following the similar episode for the at least one other patient from at least one parameter database 110. These may be certain vital sign measurements, for example, and may vary depending on the application.

Step 408 relates to the at least one patient of step 406. Step 408 involves categorizing this patient into a control group or a positive group using at least some of the retrieved action records. Categorizing the patient into the positive group may indicate the condition of the patient is at risk of deterioration. Categorizing the patient into the control group may indicate the condition of the patient is not at risk of deterioration.

Step 410 involves training at least one algorithm for deterioration detection utilizing the retrieved physiological parameters for at least one patient in the positive group. This could be an algorithm trained to use a combination of physiological parameters to detect a certain condition, for example, respiratory illness.

Alarm management strategies are an important component of deterioration detection and providing healthcare. As discussed previously, deterioration detection models may often generate a score based on patients' physiological parameters. Very essential to these types of models are alarm management strategies that determine whether to present the score as a risk indicator or an alarm. Additionally, the sensitivity and specificity of the alarm must be appropriately tailored to best serve the users. Variables in the alarm management system may include the user's work patterns, their tolerance to false alarms, and how they would like the alarm management system to be integrated into their workflow.

Presently, medical devices and software can generate various types of alarms in healthcare institutions. These alerts may be issued if, for example, it is determined that a patient has a health-related condition. Features of the present invention may monitor and record how users respond to these alarms and their sequential actions.

When an alarm is signaled by a device or other software, the user tracker module 104 may record when (i.e., t₀) and how the user responds to the alarm. For example, the user may accept or dismiss the alarm.

The alarm(s) may be communicated in a variety of ways. For example, if the user is carrying a mobile device such as device 102b of FIG. 1, an alert may be presented to the user via a written message, an auditory message, a haptic-based message, or some combination thereof. Regardless of the device(s) used, alarms may be presented in writing, graphically (e.g., with difference colors indicating severity of the alarm), or auditory.

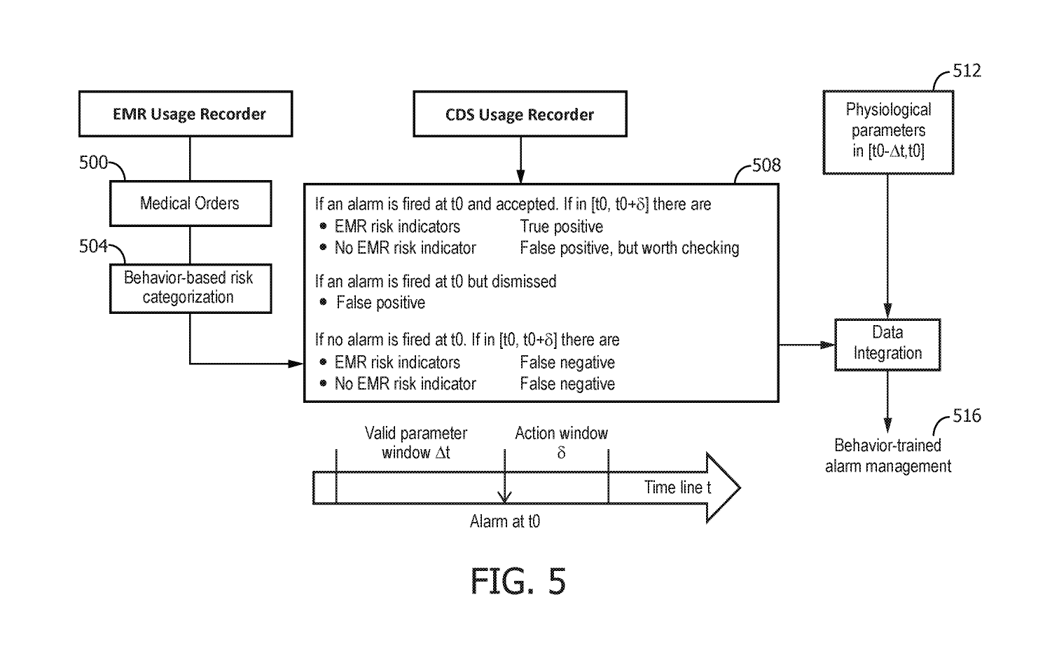

FIG. 5 generally illustrates a series of steps that may be performed by an alarm management module 116. As discussed above in connection with the deterioration detection module 114, the user tracker module 104 may track medical orders placed in an EMR by users in a δ time window [t₀, t_{+δ}]. It may be assumed that in the δ time window, users may place medical orders to treat the emergent condition signaled by the alarm as well as perform other actions (Step 500).

A series of criteria may be programmed to determine if the collection of actions taken in the time window [t₀, t_{+δ}] indicate if the patient is at risk of deterioration (or some other health-related condition) (Step 504). For example, ordering a certain dosage of a particular medication may indicate that the patient is at risk. On the other hand, conducting no further tests or ordering no medications may indicate that the patient is not at risk.

FIG. 5 illustrates a series of scenarios 508 in which an alarm is and isn't issued, and, if issued whether the alarm is accepted or dismissed. The value of the alarm can be a false positive (i.e., if the alarm is dismissed or ignored); true positive (if the alarm is accepted and followed by medical interventions that indicate there is a risk), or a false positive (if the alarm is accepted but not medical interventions are taken).

A false negative occurs when no alarm is signaled but there are medical interventions that indicate risk. A true negative occurs when no alarm is signaled and there are no actions that indicate or otherwise suggest there is a risk.

As in the deterioration detection module 114, the integrator module 112 may obtain physiological parameters in a time window before t₀ [t_{0-Δt}, t₀] (Step 512). Collectively, data from thousands of episodes of thousands of patients may be integrated.

In this application, the alarm management module 116 may use the behavior-based categorization as ground truth to separate alarms into different categories (i.e., true positive, false positive, true negative, and false negative). The alarm management module 116 also uses the physiological parameters as input to train new alarm management strategies.

As seen in FIG. 5, the output may be a new alarm system 516 in which the sensitivity and specificity of the alarm is optimized by learning users' behaviors. This outcome of the integrator could be fed back into the system, tested, and then improved by taking several iterations. The integrator module 112 could be programmed to utilize various types of data mining techniques, such as regression, decision trees, random forest, vector support machine, and Bayesian methods.

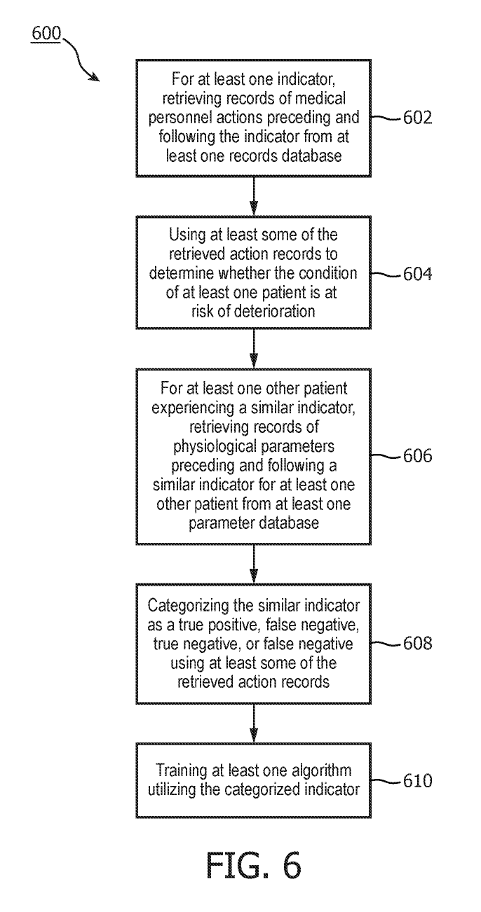

FIG. 6 depicts a flow chart of a method 600 of behavior-trained alarm management in accordance with one embodiment of the invention. For at least one indicator, step 602 involves retrieving records of medical personnel actions preceding and following the indicator from at least one records database. These records may be stored in the records database 106 of FIG. 1, for example.

Step 604 involves using at least some of the retrieved action records to determine whether the condition of the at least one patient is at risk of deterioration. This may be determined by the analyzer module 108 of FIG. 1, for example. This determination may be based on medical orders placed by users and/or other actions taken by users. For example, if a user dismissed the indicator, then it is likely the patient's condition is not at risk of deterioration.

Step 606 involves, for at least one other patient that experiences a similar indicator, retrieving records of physiological parameters preceding and following the similar indicator from at least one parameter database. These records of physiological parameters may be obtained from the parameter database 110 of FIG. 1, for example.

Step 608 involves categorizing the similar indicator as a true positive, false positive, true negative, or false negative using at least some of the retrieved action records. This categorization may be made using the analyzer module 108 of FIG. 1, for example.

Step 610 involves training at least one algorithm utilizing the categorized indicator.

This may be accomplished by the integrator module 112 after integrating information regarding the categorization and the physiological parameters.

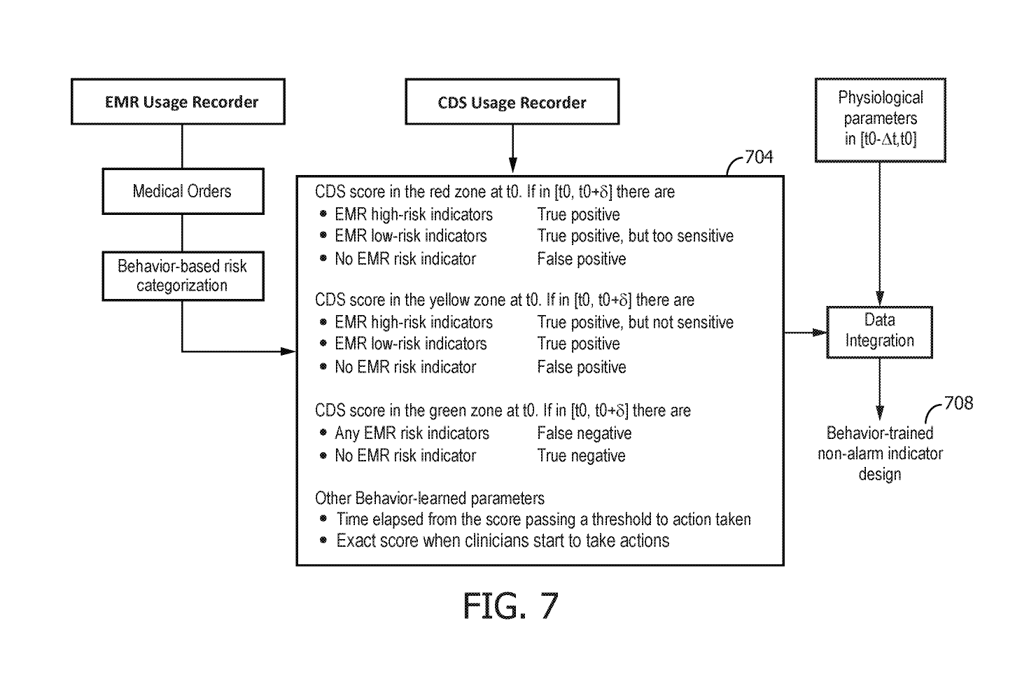

FIG. 7 generally illustrates a series of steps that may be executed by the alarm management module 116 in another embodiment of the invention. This specific embodiment may be referred to as “non-alarm indicator management.” Non-alarm indicators have become increasingly popular as they typically generate less noise and disruption in the healthcare institution while still providing risk-related information.

In this embodiment, there are no alarms in the form of sound or light. However, the non-alarm indicator may be displayed as a curve on a screen, for example. For example, the various I/O devices 102 of FIG. 1 may include an interface to present information graphically. The curve may enter into a red zone when the score passes a high threshold, a yellow zone when the score passes a medium threshold, and a green zone when the score is lower than a threshold. In this embodiment, the indicator is presented in a user-friendly way while still providing helpful information to a user.

As with the previously-discussed applications, the system tracks users' actions in a time window [t₀, t_{0+δ}] after the non-alarm indicator passes a certain threshold. Similar to the embodiment illustrated in FIG. 5, the value of the non-alarm indicator may be categorized 704 into true positive, false positive, true negative, and false negative. The system may then train a new non-alarm indicator 708 by combining the behavior-based categorization of physiological parameters.

This application can also learn more from users' specific behaviors. Non-alarm indicators are often based on continuous scores and do not force users to take action. The users' behavior therefore may reflect their judgment as to when is the best time for medical intervention.

For example, information such as the exact score when users start to take actions, along with the difference between the time when the score enters a certain “zone” and the time when users intervene may be analyzed. These parameters may be used to train a new non-alarm indicator and a new set of alarm management strategies. For example, an alarm may be communicated only when a score reaches a user-specific score (i.e., a score at which point a specific user normally takes action). These features may also be implemented in the applications and methods illustrated in FIGS. 5 and 6.

A third application is behavior-trained decision and navigation support. In this application the system 100 may again track users' movement across multiple platforms, such as EMR, imaging software, and various other CDS tools. The information obtained by tracking users' movements may be used to train the system 100 to assist in future patient treatment episodes.

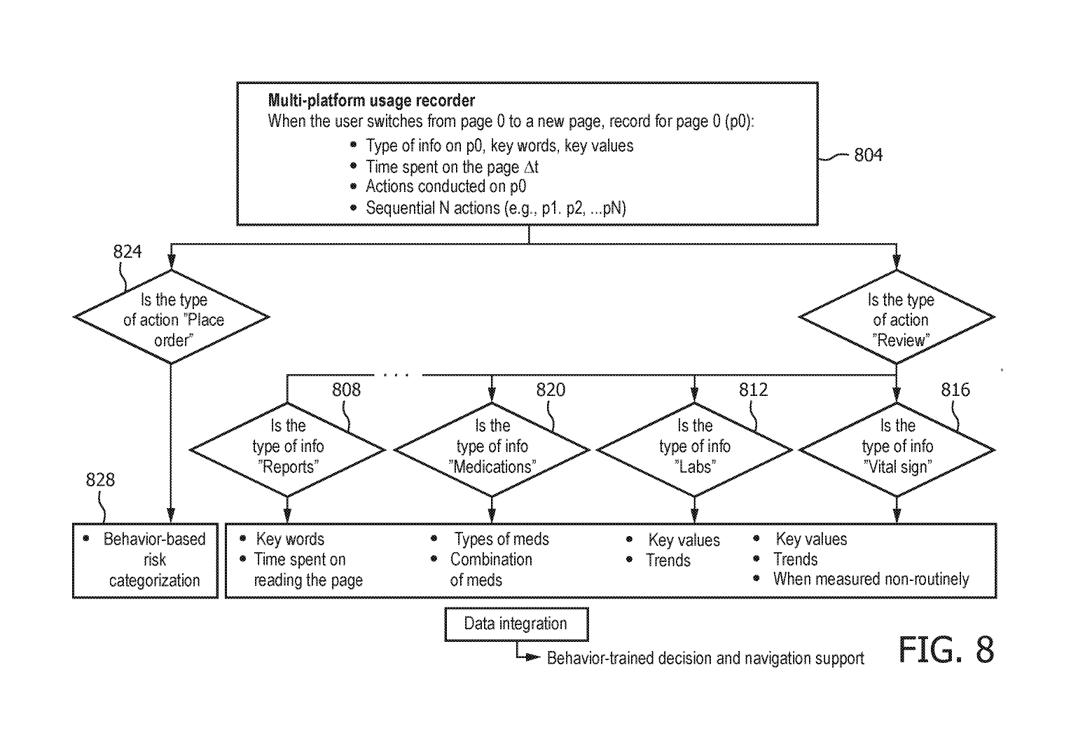

FIG. 8 graphically illustrates a series of steps taken by the decision and navigation support module 118. The user tracker module 104, for example, may monitor 804 when a user switches from one page (p0) to another page (p1), the actions taken by user on each page, words entered on each page, time spent on each page, and subsequent actions taken by the user after viewing each page. In this embodiment, a page may be an interface of software, a tab in software, or any windows that can be opened as part of a software application. These windows/tabs may be presented on an interface of a device 102.

They type of information analyzed may of course depend on the specific page the user is viewing and interacting with. For example, the user tracker module 104 may track diagnosis key words when physicians review reports 808 (e.g., progress reports, imaging reports). It may also track key values and trends of lab tests 812 when the user is viewing the EMR, vital signs and trends 816 when the user views/interacts with the vital sign tab, and combinations of medications 820 when the user views/interacts with the medication administration record tab.

The user tracker module 104 may also track medical orders placed by users 824, and the analyzer module 108 may categorize the orders by behavior-based risk categorization 828. Different types of information, collectively, from thousands of patients and users may be integrated by the integrator module 112. The system 100 thus learns the mutual relationship (e.g., timing logic relationship, physiological relationship, etc.) between various types of information and may link them to train a new decision and navigation support tool. This, among other things, may more quickly provide users with relevant information and suggest possible courses of action.

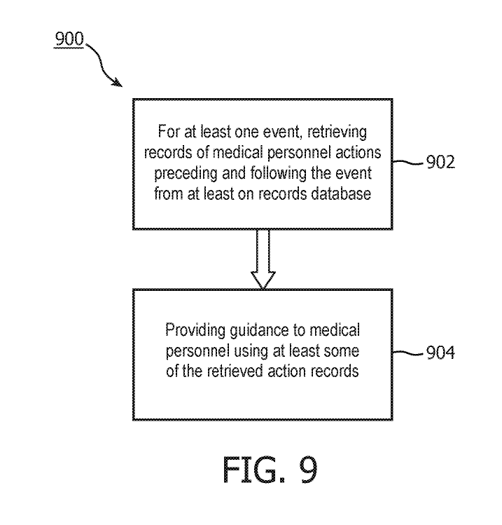

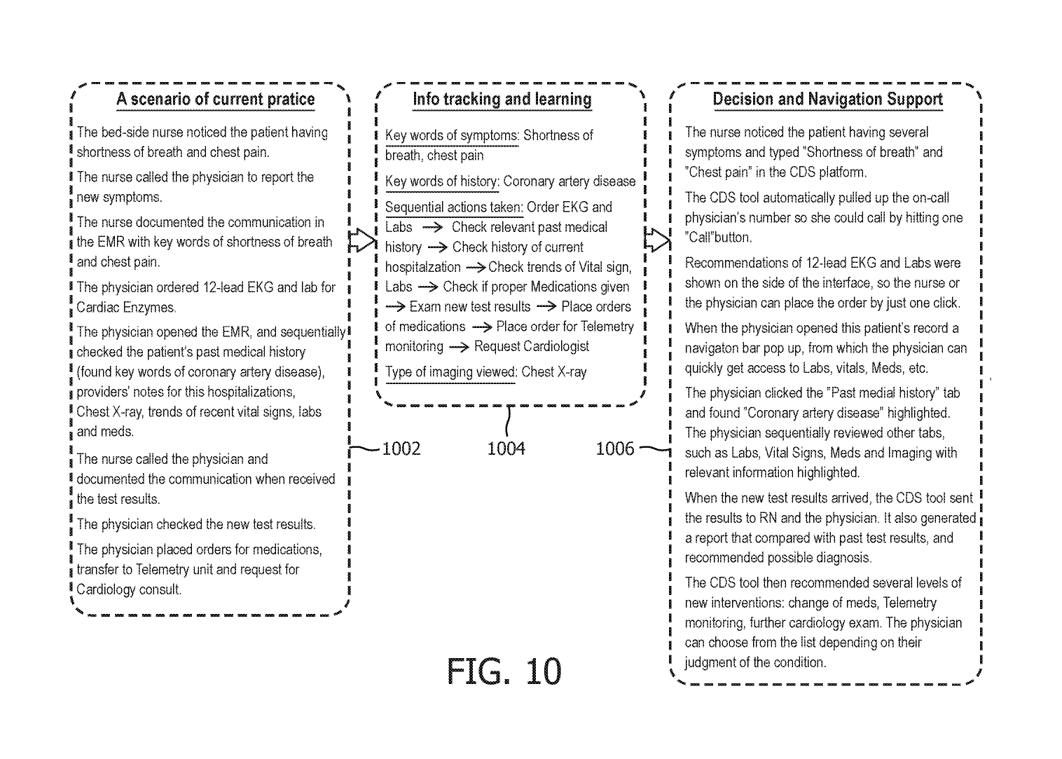

FIG. 9 depicts a flow chart of a method 900 of behavior-trained decision support. For at least one event, step 902 involves retrieving records of medical personnel actions preceding and following the event from at least one records database. These records may be obtained from the records database 106 of FIG. 1, for example.

Step 904 involves providing guidance to medical personnel using at least some of the retrieved action records. This guidance may be presented to users or other interested parties via interfaces of devices 102 of FIG. 2, for example.

FIG. 10 illustrates an exemplary application of the decision and navigation support module 118. Section 1002 illustrates a series of steps that may be made in a training exercise or in current healthcare practice when diagnosing and treating a patient. As can be seen, these steps involve a nurse making observations about a patient, documenting the observations with the key words of “shortness of breath” and “chest pain,” and calling a physician. Afterwards, the physician may study a patient's EMR and order a series of tests. The physician may then have further communication with the nurse, review tests, and order medications and/or conduct further medical interventions.

Section 1004 essentially represents information extracted by the various modules of the system 100 during the steps outlined in section 1002. In other words, the system 100 learns from users' actions from this particular scenario outlined in section 1002. This section 1004 summarizes, e.g., key words of symptoms, key words of history, actions taken, and types of imaging viewed.

Section 1006 illustrates the outcome of the decision and navigation support module 118. Specifically, section 1006 outlines a series of automated steps that assist users in treating a patient in future patient-treatment episodes. For example, say a user (nurse) notices a patient having several symptoms and typed “shortness of breath” and “chest pain” into an I/O device 102. The decision and navigation support module 118 (i.e., a CDS platform) may recognize these symptoms as indicative of a potentially serious condition that requires further attention based on the scenario outlined in section 1002.

The decision and navigation support module 118 may then automatically pull the physician's number and present a “call” button. The nurse can then quickly call the physician and update the physician regarding the observed symptoms. The decision and navigation support module 118 may also present recommendations of a 12-lead EKG via an interface of an I/O device 102, as these were the same orders that the physician made in 1002. The nurse or physician can then place the order by just one click.

When the physician opens this particular patient's EMR, a navigation bar may present the physician with tabs so the physician may quickly access to Labs, Vitals, Meds, or the like. Any test results may be uploaded (e.g., manually or automatically) to the patient's EMR, and the decision and navigation support module 118 may recommend several new medical interventions. The decision and navigation support module 118 may suggest that the physician place the patient on different medications, for example. The physician can choose form the list of suggestions depending on their judgment of the patient's condition.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims. For example, the features of the invention may be implemented in hospitals, physician offices, military camps, infirmaries, urgent care facilities, assisted living environments, maternity wards, or the like.

1. A non-transitory computer-readable medium having instructions stored thereon for performing a method of behavior-trained deterioration detection, the instructions operative on one or more processors to perform operations comprising:
retrieving records of medical personnel actions for a period preceding and a period following a medical event for a plurality of patients from at least one records database;
comparing at least some of the retrieved action records to predetermined criteria to determine whether the condition of each of the plurality of patients was likely to deteriorate;
retrieving records of physiological parameters for a period preceding and a period following the medical event for the plurality of patients from at least one parameter database;
classifying the plurality of patients into either a control group of a positive group based on whether the condition of each patient in the pluraity was determined to have been likely to deteriorate; and
training at least one algorithm for deterioration detection utilizing the retrieved physiological parameters and the classification of the patient associated with the physiological parameters as a member of the control group or the positive group.

2. The computer readable medium of claim 1 wherein the at least one records database is at least one of an electronic medical record (EMR) system and a clinical decision support (CDS) system.

3. The computer readable medium of claim 1 wherein the at least one parameter database is at least one of an electronic medical record (EMR) system and a clinical decision support (CDS) system.

4. The computer readable medium of claim 1 wherein the duration of the period preceding and the period following the medical event for the retrieval of action records depends on the type of the medical event.

5. The computer readable medium of claim 1 wherein the duration of the period preceding and the period following the medical event for the retrieval of parameter records depends on the type of the medical event.

6. The computer readable medium of claim 1 wherein the training is selected from the group consisting of regression, decision trees, random forest, vector support machine, and Bayesian methods.

7-14. (canceled)

15. The computer readable medium of claim 1 further comprising using the trained algorithm to identify a patient experiencing a similar medical event in real time.
